SEQUENCE LISTING (6 sequences)

SEQ ID NO: 1 - DNA, 99 bp, synthetic construct
  misc_feature 1..99: note = Synthetic oligonucleotide
  source 1..99: mol_type = other DNA
ttgatggtattgcacagaatatggcggcgatgctgaccggcagtgagcagaactggcgcagcttcacccgttccgtgctgtccatgatgacagaaattc

SEQ ID NO: 2 - DNA, 38 bp, synthetic construct
  misc_feature 1..38: note = 1st forward primer
  source 1..38: mol_type = other DNA
cgctcttccgatctttgatggtattgtatagaatatgg

SEQ ID NO: 3 - DNA, 38 bp, synthetic construct
  misc_feature 1..38: note = 1st reverse primer
  source 1..38: mol_type = other DNA
cgctcttccgatctaaatttctatcatcataaacaaca

SEQ ID NO: 4 - DNA, 66 bp, synthetic construct
  misc_feature 1..66: note = 2nd forward primer
  source 1..66: mol_type = other DNA
aatgatacggcgaccaccgagatctacactatagccttctttccctacacgacgctcttccgatct

SEQ ID NO: 5 - DNA, 66 bp, synthetic construct
  misc_feature 1..66: note = 2nd reverse primer
  source 1..66: mol_type = other DNA
caagcagaagacggcatacgagatcgagtaatgtgactggagttcagacgtgtgctcttccgatct

SEQ ID NO: 6 - DNA, 99 bp, synthetic construct; identical to SEQ ID NO: 1
  misc_feature 1..99: note = Synthetic oligonucleotide
  modified_base 25: mod_base = m5c
  modified_base 28: mod_base = m5c
  modified_base 38: mod_base = m5c
  modified_base 57: mod_base = m5c
  modified_base 69: mod_base = m5c
  modified_base 74: mod_base = m5c
  source 1..99: mol_type = other DNA

(19) United States
(12) Patent Application Publication
(10) Pub. No.: US 2024/0002954 A1
(43) Pub. Date: Jan. 4, 2024

(54) EVALUATION METHOD FOR BISULFITE REAGENT AND GENETIC TEST METHOD

(71) Applicant: FUJIFILM Corporation, Tokyo (JP)

(72) Inventor: Maiko WAKITA, Kanagawa (JP)

(73) Assignee: FUJIFILM Corporation, Tokyo (JP)

(21) Appl. No.: 18/357,453

(22) Filed: Jul. 24, 2023

Related U.S. Application Data
(63) Continuation of application No. PCT/JP2022/006737, filed on Feb. 18, 2022.

(30) Foreign Application Priority Data
Feb. 25, 2021 (JP) 2021-028913

(51) Int. Cl. C12Q 1/6886

(52) U.S. Cl. CPC C12Q 1/6886; C12Q 2600/118; C12Q 2600/154; C12Q 2600/166

(57) ABSTRACT

There are provided an evaluation method for selecting a bisulfite reagent according to a target of a genetic test and a genetic test method including selecting a bisulfite reagent according to a target of a genetic test. In the evaluation method for a bisulfite reagent, a rate 1 of an error in which an unmethylated CpG site is determined to be in a methylated state is compared with a rate 2 of an error in which a methylated CpG site is determined to be in an unmethylated state. In the genetic test method, the bisulfite reagent is selected according to a history of illness of a subject and a correlation between a disease and methylation or unmethylation of a gene.

### CROSS-REFERENCE TO RELATED APPLICATION

This application is a Continuation of International Application No. PCT/JP2022/006737, filed Feb. 18, 2022, which claims priority to Japanese Patent Application No. 2021-028913, filed Feb. 25, 2021. Each of the above applications is hereby expressly incorporated by reference, in its entirety, into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 21, 2023, is named “1982-1331PUS1.xml” and is 10,949 bytes in size. The sequence listing contained in this .XML file is part of the specification and is hereby incorporated by reference herein in its entirety.

### BACKGROUND OF THE INVENTION

### 1. Field of the Invention

The present disclosure relates to an evaluation method for a bisulfite reagent and a genetic test method.

### 2. Description of the Related Art

There is a phenomenon in which a methyl group is added to a carbon atom of cytosine that constitutes DNA and the DNA is methylated. It is known that methylation of DNA correlates with the onset or progression of a disease, this correlation is attracting attention as useful information for diagnosing a disease.

There are several methods of analyzing the degree of methylation of DNA, and one of the representative methods is a method in which bisulfite treatment, polymerase chain reaction (PCR), and sequence analysis are combined, that is, a bisulfite sequencing method.

JP2007-503813A discloses a method of purifying a nucleic acid sample treated with a hydrogen sulfite, by using a size exclusion technique and a size exclusion device.

JP2015-522289A discloses a method of sequencing a nucleic acid sample, which includes partial and incomplete bisulfite conversion of cytosine in a plurality of nucleic acid fragments.

### SUMMARY OF THE INVENTION

In a case where DNA is treated with a bisulfite reagent, unmethylated cytosine is converted to uracil, whereas methylated cytosine remains as cytosine. That is, by the bisulfite treatment, the modification state (the unmethylated or methylated state) of cytosine is converted into the information of sequence (uracil or cytosine) at a position thereof

However, the bisulfite reaction may cause an erroneous reaction, although being infrequent. An error in which unmethylated cytosine is not converted to uracil and remains as cytosine is called “failed conversion”. An error in which methylated cytosine is converted to uracil is called “inappropriate conversion”. In a case where the degree of methylation of DNA is used as a biomarker for a disease, the above-described two conversion errors cause a decrease in sensitivity or specificity.

JP2007-503813A describes that the hydrogen sulfite concentration is adjusted for the intended purpose of reducing failed conversion. JP2015-522289A describes that the hydrogen sulfite concentration and the reaction time are adjusted with respect to the failed conversion. However, there is a trade-off relationship between the failed conversion and the inappropriate conversion due to the bisulfite reagent, and thus in a case of reducing the failed conversion, the inappropriate conversion may increase, and in a case of reducing the inappropriate conversion, the failed conversion may increase.

The present disclosure has been made under the above circumstances.

An object of the present disclosure is to provide an evaluation method for a bisulfite reagent for selecting it according to a target of a genetic test and a genetic test method in which a bisulfite reagent selected according to a target of a genetic test is used.

Specific means for solving the above problems include the following aspects.

<1> A method of evaluating a bisulfite reagent, the method comprising the following (a) to (c):
(a) preparing a DNA sample 1 in which a CpG site to be measured is not methylated, treating the DNA sample 1 with a bisulfite reagent to be evaluated, and subsequently measuring a degree of methylation of the CpG site to be measured to calculate an error rate 1, where the error rate 1 is an average value of the degree of methylation of the CpG site to be measured;
(b) preparing a DNA sample 2 which has the same sequence as the DNA sample 1 and in which the CpG site to be measured is methylated, treating the DNA sample 2 with the bisulfite reagent to be evaluated, and subsequently measuring a degree of methylation of the CpG site to be measured to calculate an error rate 2, where the error rate 2 is calculated by 100—(an average value of the degree of methylation of the CpG site to be measured) and
(c) comparing the error rate 1 with the error rate 2, wherein when a relationship of the error rate 1≤the error rate 2 is satisfied, the bisulfite reagent is evaluated as having an inappropriate conversion tendency, and wherein when a relationship of the error rate 1>the error rate 2 is satisfied, the bisulfite reagent is evaluated as having a failed conversion tendency.

<2> A method of a genetic test which analyzes a degree of methylation of a gene of a subject who has no history of illness in regard to a target disease onset or progression of which correlates with methylation or unmethylation of the gene, the method comprising treating DNA of the subject with a bisulfite reagent,
wherein when a disease onset or progression of which correlates with methylation of the gene is the target disease, the bisulfite reagent is a bisulfite reagent which has been evaluated by the method according to <1> as having an inappropriate conversion tendency, and
wherein when a disease onset or progression of which correlates with unmethylation of the gene is the target disease, the bisulfite reagent is a bisulfite reagent which has been evaluated by the method according to <1> as having a failed conversion tendency.

<3> A method of a genetic test which analyzes a degree of methylation of a gene of a subject who has a history of illness in regard to a target disease onset or progression of which correlates with methylation or unmethylation of the gene, the method comprising treating DNA of the subject with a bisulfite reagent,
wherein when a disease onset or progression of which correlates with methylation of the gene is the target disease, the bisulfite reagent is a bisulfite reagent which has been evaluated by the method according to <1> as having a failed conversion tendency, and
wherein when a disease onset or progression of which correlates with unmethylation of the gene is the target disease, the bisulfite reagent is a bisulfite reagent which has been evaluated by the method according to <1> as having an inappropriate conversion tendency.

<4> The method according to <2> or <3>, wherein a false positive rate and a false negative rate in the test are estimated from values of the error rate 1 and the error rate 2 in the evaluation of the bisulfite reagent.

<5> The method according to any one of <2> to <4>, wherein the disease is cancer.

According to the present disclosure, there are provided an evaluation method for a bisulfite reagent for selecting it according to a target of a genetic test and a genetic test method in which a bisulfite reagent selected according to a target of a genetic test is used.

### DESCRIPTION OF THE PREFERRED EMBODIMENTS

Hereinafter, embodiments according to the present disclosure will be described. These descriptions and Examples are only illustrative of the embodiments and do not limit the ranges of the embodiments.

The term “step” in the present disclosure not only includes an independent step but also includes a step that may not be clearly distinguished from the other step but still achieves a desired effect of the step.

In the present disclosure, a numerical range expressed using “to” indicates a range including numerical values before and after “to” as a minimum value and a maximum value.

In the numerical ranges described stepwise in the present disclosure, the upper limit value or the lower limit value described in one numerical range may be replaced with the upper limit value or the lower limit value of the numerical range described stepwise in other stages. Further, in the numerical ranges described in the present disclosure, the upper limit value or the lower limit value of the numerical range may be replaced with the value shown in Examples.

In the present disclosure, the sequencer is a term including a first generation sequencer (a capillary sequencer), a second generation sequencer (a next generation sequencer), a third generation sequencer, a fourth generation sequencer, and a sequencer to be developed in the future. The sequencer may be a capillary sequencer, may be a next generation sequencer, or may be another sequencer. The sequencer is preferably a next generation sequencer from the viewpoints of the speed of analysis, the large number of specimens that can be processed at one time, and the like. The next generation sequencer (NGS) refers to a sequencer that is classified by being contrasted with a capillary sequencer (called a first generation sequencer) using the Sanger method. At present, the most popular next generation sequencer is a sequencer of which the principle is to capture fluorescence or luminescence linked to a complementary strand synthesis by DNA polymerase or a complementary strand binding by DNA ligase and determine the base sequence. Specific examples thereof include MiSeq (Illumina, Inc.), HiSeq 2000 (Illumina, Inc., HiSeq is a registered trade name), and Roche 454 (Roche, Ltd.).

In the present disclosure, methylation of cytosine refers to the addition of a methyl group to the carbon at the 5-position of the pyrimidine ring of cytosine.

<Evaluation Method for Bisulfite Reagent>

The bisulfite reagent may cause two kinds of erroneous conversions, although being infrequent. One is the failed conversion in which unmethylated cytosine is not converted to uracil and remains as cytosine. The other is the inappropriate conversion in which methylated cytosine is converted to uracil.

In a case where the failed conversion occurs, the unmethylated cytosine is erroneously determined to be in the methylated state, and the degree of methylation of DNA is measured to be higher than the original value.

In a case where the inappropriate conversion occurs, the methylated cytosine is erroneously determined to be in the unmethylated state, and the degree of methylation of DNA is measured to be lower than the original value.

The inventors of the present invention found that in a case where the degree of methylation of DNA is used as a biomarker, the above-described two kinds of conversion errors are respectively used as a means for reducing a methylation signal or a means for amplifying a methylation signal.

In addition, the inventors of the present invention found that the bisulfite reagent includes a reagent that is likely to cause failed conversion (referred to as a “reagent having a failed conversion tendency”) and a reagent that is likely to cause inappropriate conversion (referred to as a “reagent having an inappropriate conversion tendency”).

In addition, the inventors of the present invention found that the bisulfite reagent can be selected according to the properties of genetic tests (for example, a genetic test in which the sensitivity is prioritized and a genetic test in which the specificity is prioritized).

The evaluation method for a bisulfite reagent according to the present disclosure includes the following (a) to (c).
(a) Preparing a DNA sample 1 in which a CpG site to be measured is not methylated, treating the DNA sample 1 with a bisulfite reagent to be evaluated, and subsequently measuring a degree of methylation of the CpG site to be measured to calculate an error rate 1. Here, the error rate 1 is the average value of the degree of methylation of the CpG site to be measured.
(b) Preparing a DNA sample 2 which has the same sequence as the DNA sample 1 and in which the CpG site to be measured is methylated, treating the DNA sample 2 with the bisulfite reagent to be evaluated, and subsequently measuring a degree of methylation of the CpG site to be measured to calculate an error rate 2. Here, the error rate 2 is 100—(the average value of the degree of methylation of the CpG site to be measured).
(c) Comparing the error rate 1 with the error rate 2 to evaluate the bisulfite reagent to be evaluated as having an inappropriate conversion tendency in a case where the error rate 1<the error rate 2 is satisfied or to evaluate the bisulfite reagent to be evaluated as having a failed conversion tendency in a case where the error rate 1>the error rate 2 is satisfied.

Hereinafter, elements of the evaluation method for a bisulfite reagent will be described in detail.

[Bisulfite Reagent]

The bisulfite reagent is a reagent that converts unmethylated cytosine in a base sequence to uracil by a bisulfite reaction, and where it is a reagent containing a hydrogen sulfite as a main component.

The bisulfite reagent is used in a bisulfite sequencing method. The outline of the bisulfite sequencing method is as follows.

In a case where DNA is treated with a bisulfite reagent, unmethylated cytosine is converted to uracil, whereas methylated cytosine remains as cytosine. That is, by the bisulfite treatment, the modification state (the unmethylated or methylated state) of cytosine is converted into the information of sequence (uracil or cytosine) at a position thereof. Next, DNA is amplified according to polymerase chain reaction (PCR). In this process, uracil is converted to thymine. Next, the sequence of the amplification product is analyzed using a sequencer. By determining whether the base at the position to be analyzed is thymine or cytosine, it is possible to know the modification state (the unmethylated or methylated state) of cytosine at the position of interest in DNA.

The bisulfite reagent to be evaluated may be a commercially available product or a newly prepared reagent. Examples of the commercially available product of the bisulfite reagent include an EpiTect Plus Bisulfite Conversion Kit (manufactured by Qiagen N.V.), an EpiTect Fast DNA Bisulfite Kit (manufactured by Qiagen N.V.), an EZ DNA Methylation Direct Kit (manufactured by Zymo Research Corporation), an EZ DNA Methylation Gold Kit (manufactured by Zymo Research Corporation), an EZ DNA Methylation Lightning Kit (Zymo Research Corporation), an innuCONVERT Bisulfite Body Fluids Kit (Analytik Jena AG), an innuCONVERT Bisulfite Basic Kit (Analytik Jena AG), and Premium Bisulfite (Diagenode Inc.).

In a case where the bisulfite reagent is a commercially available product, it is preferable that the treatment of the DNA sample 1 and the DNA sample 2 with the bisulfite reagent follows the recommended protocol of the bisulfite reagent.

DNA is subjected to treatment with a bisulfite reagent by being reacted at a high molar concentration and a high temperature for a relatively short time or by being reacted at a lower molar concentration and a lower temperature for a relatively long time. In general, a reaction time of less than 8 hours corresponds to the former, and a reaction time of 8 hours or more corresponds to the latter.

[Degree of Methylation of CpG Site]

The CpG site is a two-base sequence in which guanine appears next to cytosine.

The degree of methylation of a CpG site is a value calculated from a set of DNA fragments and is calculated for each CpG site. A degree of methylation of a certain CpG site is {the number of DNA fragments in which the CpG site is methylated/(the number of DNA fragments in which the CpG site is methylated+the number of DNA fragments in which the CpG site is unmethylated)}, and it is indicated in terms of percentage (%).

In a case where DNA has a plurality of CpG sites to be measured, the degree of methylation of the CpG sites to be measured is a value obtained by averaging the degrees of methylation of the respective CpG sites.

[DNA sample 1 and DNA sample 2]

The DNA sample 1 and the DNA sample 2 have the same base sequence; however, they differ in the modification state (the unmethylated or methylated state) of the CpG site to be measured.

The DNA sample 1 and the DNA sample 2 may be a synthetic DNA or a standard DNA.

In the DNA sample 1, the cytosine at the CpG site to be measured is in an unmethylated state. The DNA sample 1 is defined as DNA in which the degree of methylation is 0% to 5% according to a general methylation measurement technique.

In the DNA sample 2, the cytosine at the CpG site to be measured is in a methylated state. The DNA sample 2 is defined as DNA in which the degree of methylation is 95% to 100% according to a general methylation measurement technique.

The base length of each of the DNA sample 1 and the DNA sample 2 is preferably 50 bases to 150 bases, more preferably 50 bases to 125 bases, and still more preferably 50 bases to 100 bases.

The number of CpG sites to be measured which are included in each of the DNA sample 1 and the DNA sample 2 is preferably 1 to 10, more preferably 2 to 8, and still more preferably 3 to 6.

[Error rate 1 and error rate 2]

The error rate 1 is, in short, a rate by which the unmethylated CpG site is determined to be in the methylated state. The error rate 1 is determined by treating the DNA sample 1 with a bisulfite reagent to be evaluated and then carrying out PCR and sequence analysis. The bisulfite treatment, PCR, and sequence analysis of the DNA sample 1 are carried out three times, and a value (%) obtained by averaging the degrees of methylation from the three measurements is defined as an error rate 1 (%).

The error rate 2 is, in short, a rate by which the methylated CpG site is determined to be in the unmethylated state. The error rate 2 is determined by treating the DNA sample 2 with a bisulfite reagent to be evaluated and then carrying out PCR and sequence analysis. The bisulfite treatment, PCR, and sequence analysis of the DNA sample 2 are carried out three times, and a value (%) obtained by averaging the degrees of methylation from the three measurements is subtracted from 100 to obtain a value which is defined as an error rate of 2 (%).

[Inappropriate Conversion Tendency and Failed Conversion Tendency]

The error rate 1 is compared with the error rate 2 to evaluate the bisulfite reagent to be evaluated as having an inappropriate conversion tendency in a case where the error rate 1<the error rate 2 is satisfied. In a case where the inappropriate conversion occurs, the methylated cytosine is erroneously determined to be in the unmethylated state, and the degree of methylation of DNA is measured to be lower than the original value. The bisulfite reagent having an inappropriate conversion tendency is such a bisulfite reagent that the possibility that the degree of methylation of DNA is measured to be lower than the original value is higher than the possibility that the degree of methylation of DNA is measured to be higher than the original value. The bisulfite reagent having an inappropriate conversion tendency can be said to be a means for reducing a methylation signal.

The error rate 1 is compared with the error rate 2 to evaluate the bisulfite reagent to be evaluated as having a failed conversion tendency in a case where the error rate 1>the error rate 2 is satisfied. In a case where the failed conversion occurs, the unmethylated cytosine is erroneously determined to be in the methylated state, and the degree of methylation of DNA is measured to be higher than the original value. The bisulfite reagent having a failed conversion tendency is such a bisulfite reagent that the possibility that the degree of methylation of DNA is measured to be higher than the original value is higher than the possibility that the degree of methylation of DNA is measured to be lower than the original value. The bisulfite reagent having a failed conversion tendency can be said to be a means for amplifying a methylation signal.

In a case where the bisulfite reagent is a commercially available product, the following points can be said generally. A bisulfite reagent having a recommended protocol in which the reaction time is 8 hours or more is likely to be evaluated as a bisulfite reagent having an inappropriate conversion tendency. A bisulfite reagent having a recommended protocol in which the reaction time is less than 8 hours is likely to be evaluated as a bisulfite reagent having a failed conversion tendency. As a result, the recommended protocol makes it possible to predict whether the bisulfite reagent has an inappropriate conversion tendency or a failed conversion tendency before carrying out the evaluation method for a bisulfite reagent according to the present disclosure.

<Genetic Test Method>

The subject of the genetic test method according to the present disclosure is a human. The subject is, for example, an examinee who has undergone a medical examination according to his/her own will, a person who has been suspected of having a disease at a medical institution, a patient who is being on medical treatment, or a former patient who has recovered from a disease.

The information obtained by the genetic test method according to the present disclosure is useful as information that assists the diagnosis of a doctor, a basis for a doctor or a subject to determine whether a detailed test (for example, an imaging test) is necessary, a basis for a doctor to select a medical treatment method or a therapeutic drug, a motivation for a subject to improve his/her lifestyle habit, or the like.

The genetic test method according to the present disclosure is a genetic test method for analyzing a degree of methylation of a gene. The disease as a test target in the genetic test method according to the present disclosure is not particularly limited as long as it is a disease associated with methylation or unmethylation of cytosine in a gene. Examples of the disease include cancer, autoimmune disease, neurological disease, heart disease, cardiovascular disease, cerebrovascular disease, metabolic disease, and endocrine disease. Examples of the cancer include lung cancer, esophageal cancer, gastric cancer, colon cancer, pancreatic cancer, hepatocellular cancer, gallbladder cancer, bile duct cancer, kidney cancer, bladder cancer, urinary tract cancer, breast cancer, ovarian cancer, cervical cancer, prostate cancer, skin cancer, leukemia, myeloma, lymphoma, thyroid cancer, and brain tumor. Examples of diseases other than cancer include rheumatoid arthritis and schizophrenia.

In the genetic test method according to the present disclosure, a disease of which onset or progression correlates with methylation or unmethylation of a gene is set as a detection target, and a degree of methylation of the gene is analyzed. In the genetic test method according to the present disclosure, the bisulfite reagent is selected according to the history of illness of the subject and the correlation between the disease and the methylation or unmethylation of the gene. The present disclosure discloses two embodiments as the genetic test method.

In a first embodiment, a person having no history of illness in regard to a disease as a detection target is set as a subject. The first embodiment includes treating DNA of the subject with a bisulfite reagent, where in a case where a disease of which onset or progression correlates with methylation of a gene is set as a detection target, a bisulfite reagent evaluated as having an inappropriate conversion tendency by the evaluation method for a bisulfite reagent according to the present disclosure is used, and in a case where a disease of which onset or progression correlates with unmethylation of a gene is set as a detection target, a bisulfite reagent evaluated as having a failed conversion tendency by the evaluation method for a bisulfite reagent according to the present disclosure is used.

In a second embodiment, a person having a history of illness in regard to a disease as a detection target is set as a subject. The second embodiment includes treating DNA of the subject with a bisulfite reagent, where in a case where a disease of which onset or progression correlates with methylation of a gene is set as a detection target, a bisulfite reagent evaluated as having a failed conversion tendency by the evaluation method for a bisulfite reagent according to the present disclosure is used, and in a case where a disease of which onset or progression correlates with unmethylation of a gene is set as a detection target, a bisulfite reagent evaluated as having an inappropriate conversion tendency by the evaluation method for a bisulfite reagent according to the present disclosure is used.

By selecting the bisulfite reagent as described above, the genetic test method according to the present disclosure can be appropriate according to the properties of the genetic test (for example, a genetic test in which the specificity is prioritized and a genetic test in which the sensitivity is prioritized) without making significant changes in the test process.

Hereinafter, elements of the genetic test method will be described in detail.

The “disease of which onset or progression correlates with methylation of a gene” refers to a disease in which a CpG site of a certain gene is in an unmethylated state in a healthy state and becomes to be in a methylated state in association with the onset or progression of the disease. For example, esophageal cancer that correlates with the methylation of the metallothionein 1M (MT1M) gene corresponds thereto. The MT1M gene is in an unmethylated state in a healthy state but is in a methylated state in the cancer tissues of a large number of esophageal cancer patients.

The “disease of which onset or progression correlates with unmethylation of a gene” refers to a disease in which a CpG site of a certain gene is in a methylated state in a healthy state and becomes to be in an unmethylated state in association with the onset or progression of the disease. For example, hepatocellular cancer that correlates with the unmethylation of the long interspersed nucleotide element 1 (LINE-1) gene corresponds thereto. The LINE-1 gene is in a methylated state in a healthy state but is in an unmethylated state in the cancer tissues of a large number of hepatocellular cancer patients.

In a case where the degree of methylation of DNA is used as a biomarker for a disease, the false positivity in a disease of which onset or progression correlates with methylation of a gene means that unmethylated cytosine is detected as being in a methylated state.

In a case where the degree of methylation of DNA is used as a biomarker for a disease, the negativity positivity in a disease of which onset or progression correlates with methylation of a gene means that methylated cytosine is detected as being in an unmethylated state.

In a case where the degree of methylation of DNA is used as a biomarker for a disease, the false positivity in a disease of which onset or progression correlates with unmethylation of a gene means that methylated cytosine is detected as being in an unmethylated state.

In a case where the degree of methylation of DNA is used as a biomarker for a disease, the false negativity in a disease of which onset or progression correlates with unmethylation of a gene means that unmethylated cytosine is detected as being in a methylated state.

The “person having no history of illness in regard to diseases” is, for example, an examinee who has undergone a medical examination according to his/her own will. In the medical examination, it should be avoided to wrongly diagnosis a healthy person as an affected person. In addition, since medical examinations are generally carried out on a relatively large scale (for example, in Japan, hundreds of thousands of people per year are expected as examinees for the primary screening for cancer), wrong diagnosis is not desirable either from the viewpoint of the medical economy for suppressing the cost required for the secondary screening (for example, imaging test).

Therefore, in a case where the subject is a person having no history of illness in regard to diseases, the genetic test preferably has a high specificity, that is, it preferably has a high true negative rate, in other words, it is preferably has a low false positive rate. As a result, the selection of the following (1) and (2) are derived as preferred forms in a case where the subject is a person having no history of illness in regard to diseases.
In case where subject is person having no history of illness in regard to diseases
(1) In a case where the detection target is a disease of which onset or progression correlates with methylation of a gene, a bisulfite reagent in which the possibility that the degree of methylation of DNA is measured to be lower than the original value is higher than the possibility that the degree of methylation of DNA is measured to be higher than the original value is used. That is, a bisulfite reagent evaluated as having an inappropriate conversion tendency is used. In other words, the conversion error of the bisulfite reagent is used as a means for reducing a methylation signal.
(2) In a case where the detection target is a disease of which onset or progression correlates with unmethylation of a gene, a bisulfite reagent in which the possibility that the degree of methylation of DNA is measured to be higher than the original value is higher than the possibility that the degree of methylation of DNA is measured to be lower than the original value is used. That is, a bisulfite reagent evaluated as having a failed conversion tendency is used. In other words, the conversion error of the bisulfite reagent is used as a means for amplifying a methylation signal.

The “person having a history of illness in regard to diseases” is, for example, a patient who is being on medical treatment or a former patient who has recovered from a disease. In a monitoring test or follow-up test of a patient or former patient, patient oversight should be avoided.

Therefore, in a case where the subject is a person having a history of illness in regard to diseases, the genetic test preferably has a high sensitivity, that is, it preferably has a high true positive rate, in other words, it is preferably has a low false negative rate. As a result, the selection of the following (3) and (4) are derived as preferred forms in a case where the subject is a person having a history of illness in regard to diseases.

In case where subject is person having history of illness in regard to diseases
(3) In a case where the detection target is a disease of which onset or progression correlates with methylation of a gene, a bisulfite reagent in which the possibility that the degree of methylation of DNA is measured to be higher than the original value is higher than the possibility that the degree of methylation of DNA is measured to be lower than the original value is used. That is, a bisulfite reagent evaluated as having a failed conversion tendency is used. In other words, the conversion error of the bisulfite reagent is used as a means for amplifying a methylation signal.
(4) In a case where the detection target is a disease of which onset or progression correlates with unmethylation of a gene, a bisulfite reagent in which the possibility that the degree of methylation of DNA is measured to be lower than the original value is higher than the possibility that the degree of methylation of DNA is measured to be higher than the original value is used. That is, a bisulfite reagent evaluated as having an inappropriate conversion tendency is used. In other words, the conversion error of the bisulfite reagent is used as a means for reducing a methylation signal.

In the genetic test method according to the present disclosure, DNA, which is a specimen for analyzing a degree of methylation of a gene, is obtained from a biological specimen of a subject. The biological specimen is, for example, tissue, blood cells, blood, lymph, urine, feces, saliva, tears, cerebrospinal fluid, pericardial fluid, pleural effusion, or ascites.

The biological specimen is preferably selected according to the kind of disease to be tested. In a case where the test target is cancer, the biological specimen is preferably blood, urine, feces, saliva, cerebrospinal fluid, pericardial fluid, pleural effusion, or ascites. It is known that circulating tumor DNA (ctDNA) released from cancer cells or tumor cells is present in the blood, urine, feces, saliva, cerebrospinal fluid, pericardial fluid, pleural effusion, or ascites, which is a very versatile biological specimen for a plurality of kinds of cancer. The biological specimen is preferably blood, urine, feces, or saliva from the viewpoint of low invasiveness to a subject, and it is preferably blood from the viewpoint that the concentration of ctDNA is relatively high and from the viewpoint that ctDNA of various kinds of cancers can be contained.

In the present disclosure, the blood includes blood itself and blood diluted with physiological saline; stored blood obtained by adding additives such as glucose and an anticoagulant agent to blood; fractions thereof (for example, blood plasma and serum); and the like.

The extraction of the DNA from the biological specimen may be carried out by extracting DNA from cells contained in the biological specimen or may be carried out by extracting cell-free DNA contained in the biological specimen.

The degree of methylation of a gene is analyzed by the bisulfite sequencing method. In the bisulfite sequencing method, a bisulfite reagent having an inappropriate conversion tendency or a bisulfite reagent having a failed conversion tendency, which is selected according to the target of the genetic test, is used.

### EXAMPLES

Hereinafter, embodiments of the invention will be further described with reference to Examples. However, the embodiments of the invention are not limited to these Examples.

### Example 1: Evaluation of Bisulfite Reagent

### Preparation of DNA Sample 1-1

As an example of an embodiment of the DNA sample 1, a DNA having a sequence of SEQ ID NO: 1 was synthesized. In this DNA, cytosines at the 25th, 28th, 38th, 57th, 69th, and 74th positions from the 5′ terminal correspond to CpG sites, where all the CpG sites are not methylated. Hereinafter, this DNA is referred to as a DNA sample 1-1.

DNA sample 1-1
(SEQ ID NO: 1)
TTGATGGTATTGCACAGAATATGGCGGCGATGCTGACCGGCAGTGAGCAG
AACTGGCGCAGCTTCACCCGTTCCGTGCTGTCCATGATGACAGAAATTC

[Bisulfate Treatment of DNA Sample 1-1]

The DNA sample 1-1 was treated with an EZ DNA Methylation Gold Kit (manufactured by Zymo Research Corporation), which is a bisulfite reagent. The bisulfite treatment was carried out according to the recommended protocol of this product.

[Measurement of Degree of Methylation of DNA Sample 1-1]

Using a primer having a sequence of SEQ ID NO: 2 as a forward primer and a primer having a sequence of SEQ ID NO: 3 as a reverse primer, the DNA sample 1-1 after the bisulfite treatment was amplified by multiplex PCR. Multiplex PCR was carried out using KOD-Multi & Epi-(manufactured by TOYOBO Co., Ltd.) according to the instruction manual of this product. 25 μL of 2X PCR Buffer for KOD-Multi & Epi-, 1 μL of KOD-Multi & Epi-, 15 μL of 1 μM primer mix, 8.5 μL of the DNA after the bisulfite treatment, and 0.5 μL of water was dispensed into a PCR tube. Regarding PCR, one cycle of 94° C./2 minutes was carried out, and then three steps of 98° C./10 seconds, 58° C./30 seconds, and 68° C./15 seconds were repeated by 40 cycles. The amplification reaction solution was purified using AMPure XP (manufactured by Beckman Coulter, Inc.), and the purified DNA was recovered in 40 μL of a Tris-EDTA buffer solution.

Forward primer
(SEQ ID NO: 2)
cgctcttccgatctTTGATGGTATTGTATAGAATATGG
Reverse primer
(SEQ ID NO: 3)
cgctcttccgatctAAATTTCTATCATCATAAACAACA

Using the recovered DNA, a primer having a sequence of SEQ ID NO: 4 as a forward primer (manufactured by Fasmac Co., Ltd.), and a primer having a sequence of SEQ ID NO: 5 as a reverse primer (manufactured by Fasmac Co., Ltd.), DNA was amplified by index addition PCR. Index addition PCR was carried out using a Multiplex PCR Assay Kit (manufactured by Takara Bio Inc.). 1 μL of each of 1.25 μL primers, 0.125 μL of Multiplex PCR Mix 1, 12.5 μL of Multiplex PCR Mix 2, were used, and the final liquid volume was adjusted to 25 μL of water to prepare a reaction solution. Regarding PCR, one cycle of 94° C./3 minutes was carried out, and then three steps of 94° C./45 seconds, 50° C./60 seconds, and 72° C./30 seconds were repeated by 5 cycles, and three steps of 94° C./45 seconds, 55° C./60 seconds, and 72° C./30 seconds were repeated by 11 cycles.

Forward primer
(SEQ ID NO: 4)
AATGATACGGCGACCACCGAGATCTACACtatagcctTCTTTCCCTACA
CGACGCTCTTCCGATCT
Reverse primer
(SEQ ID NO: 5)
CAAGCAGAAGACGGCATACGAGATcgagtaatGTGACTGGAGTTCAGAC
GTGTGCTCTTCCGATCT

The obtained PCR product was purified using an AMPure XP Kit (manufactured by Beckman Coulter, Inc.). The concentration of the purified DNA was quantified using BioAnalyzer (manufactured by Agilent Technologies, Inc.) and more accurately quantified using a KAPA Library Quantification Kit (manufactured by KAPA Biosystems, Inc.). The purified DNA was used as a specimen and sequenced using a Miseq Reagent Kit v2 300 Cycle (manufactured by Illumina, Inc.). The information on the degree of methylation was acquired by mapping the obtained FastQ file to a human genome sequence using Bismark. In this way, the degree of methylation of the CpG site at each of the six positions in the DNA sample 1-1 was obtained.

The procedure from [Bisulfate treatment of DNA sample 1-1] to [Measurement of degree of methylation of DNA sample 1-1] was carried out three times.

[Calculation of Error Rate 1]

As a result of averaging the degrees of methylation of CpG sites at six positions at each time of the procedure and averaging the degrees of methylation at three times of the procedure, the average value of the degree of methylation was 0.5%. This value was defined as the error rate 1.

[Preparation of DNA sample 2-1]

As an example of an embodiment of the DNA sample 2, a DNA having a sequence of SEQ ID NO: 6 was synthesized.

This DNA has the same sequence as DNA sample 1-1; however, all CpG sites are methylated (that is, cytosines at the 25th, 28th, 38th, 57th, 69th, and 74th positions from the 5′ terminal are methylated).

Hereinafter, this DNA is referred to as a DNA sample 2-1.

DNA sample 2-1
(SEQ ID NO: 6)
TTGATGGTATTGCACAGAATATGG[5MedC]GG[5MedC]GATGCTGAC
[5MedC]GGCAGTGAGCAGAACTGG[5MedC]GCAGCTTCACC
[5MedC]GTTC[5MedC] GTGCTGTCCATGATGACAGAAATTC
(here, [5MedC] indicates methylated cytosine)

[Bisulfite Treatment of DNA Sample 2-1]

The DNA sample 2-1 was treated with an EZ DNA Methylation Gold Kit (manufactured by Zymo Research Corporation) in the same manner as in the treatment in [Bisulfite treatment of DNA sample 1-1].

[Measurement of Degree of Methylation of DNA Sample 2-1]

The same process as [Measurement of degree of methylation of DNA sample 1-1] was carried out to obtain the degree of methylation of the CpG site at each of the six positions in the DNA sample 2-1.

The procedure from [Bisulfite treatment of DNA sample 2-1] to [Measurement of degree of methylation of DNA sample 2-1] was carried out three times.

[Calculation of Error Rate 2]

As a result of averaging the degrees of methylation of CpG sites at six positions at each time of the procedure and averaging the degrees of methylation at three times of the procedure, the average value of the degree of methylation was 97.6%. From this value, the error rate 2 was calculated to be 2.4%.

[Evaluation of Bisulfite Reagent]

In the EZ DNA Methylation Gold Kit (manufactured by Zymo Research Corporation) which is a bisulfite reagent, the error rate 2=2.4% was larger than the error rate 1=0.5%.

As a result, this bisulfite reagent was evaluated as having an inappropriate conversion tendency.

[Evaluation of Another Bisulfite Reagent]

Regarding the EpiTect Plus Bisulfite Conversion Kit (manufactured by Qiagen N.V.) as well, which is a bisulfite reagent, in a case of carrying out the same process as described above using DNA sample 1-1 and DNA sample 2-1, the obtained results were the error rate 1=1.3% and the error rate 2=1.2%.

The error rate 2 was smaller than the error rate 1. As a result, this bisulfite reagent was evaluated as having a failed conversion tendency.

### Example 2: Genetic Test for Cancer

The morbidity rate of cancer in people in their 40s in Japan is about 0.05% (about 50 persons per 100,000 population). From this, it is estimated that among 100,000 people in their 40s who have no history of illness in regard to cancer, 50 potentially cancer-affected patients are present and 99,950 people do not have cancer.

[Selection of Test Target]

A cancer screening test is carried out on 100,000 subjects in their 40s who have no history of illness in regard to cancer. A cancer of which onset or progression correlates with methylation of a gene is set as the cancer as a test target.

[Genetic Test Using Bisulfite Reagent Having Inappropriate Conversion Tendency]

Using the EZ DNA Methylation Gold Kit (manufactured by Zymo Research Corporation), which is a bisulfite reagent having an inappropriate conversion tendency, the degree of DNA methylation of a subject is analyzed regarding the test target gene.

In the EZ DNA Methylation Gold Kit, the error rate 1 is 0.5%, and the error rate 2 is 2.4%. As a result, in a case carrying out bisulfite sequencing using the EZ DNA Methylation Gold Kit, the results can be 499 persons of false positive (99,950×0.5%) and one person of false negative (50×2.4%).

[Genetic Test Using Bisulfite Reagent Having Failed Conversion Tendency]

Using the EpiTect Plus Bisulfite Conversion Kit (manufactured by Qiagen N. V.) having a failed conversion tendency, which is a bisulfite reagent, the degree of DNA methylation of a subject is analyzed regarding the test target gene.

In the EpiTect Plus Bisulfite Conversion Kit, the error rate 1 is 1.3%, and the error rate 2 is 1.2%. As a result, in a case carrying out bisulfite sequencing using the EpiTect Plus Bisulfite Conversion Kit, the results can be 1,299 persons of false positive (99,950×1.3%) and zero person of false negative (50×1.2%).

In the primary screening carried out on healthy subjects on a relatively large scale, it is preferable that the specificity is high, that is, it is preferable that the false positive rate is low. In addition, in a case where a cancer of which onset or progression correlates with methylation of a gene is a detection target, a bisulfite reagent evaluated as having an inappropriate conversion tendency is preferable as compared with a bisulfite reagent evaluated as having a failed conversion tendency as the bisulfite reagent for treating DNA of a subject.

[Sequence List]

International application based on the International Patent Cooperation Treaty 21F00131W1JP22006737_0.app

What is claimed is:

1. A method of evaluating a bisulfite reagent, the method comprising the following (a) to (c):
(a) preparing a DNA sample 1 in which a CpG site to be measured is not methylated, treating the DNA sample 1 with a bisulfite reagent to be evaluated, and subsequently measuring a degree of methylation of the CpG site to be measured to calculate an error rate 1, where the error rate 1 is an average value of the degree of methylation of the CpG site to be measured;
(b) preparing a DNA sample 2 which has the same sequence as the DNA sample 1 and in which the CpG site to be measured is methylated, treating the DNA sample 2 with the bisulfite reagent to be evaluated, and subsequently measuring a degree of methylation of the CpG site to be measured to calculate an error rate 2, where the error rate 2 is calculated by 100—(an average value of the degree of methylation of the CpG site to be measured) and
(c) comparing the error rate 1 with the error rate 2, wherein when a relationship of the error rate 1≤the error rate 2 is satisfied, the bisulfite reagent is evaluated as having an inappropriate conversion tendency, and wherein when a relationship of the error rate 1>the error rate 2 is satisfied, the bisulfite reagent is evaluated as having a failed conversion tendency.

2. A method of a genetic test which analyzes a degree of methylation of a gene of a subject who has no history of illness in regard to a target disease onset or progression of which correlates with methylation or unmethylation of the gene, the method comprising treating DNA of the subject with a bisulfite reagent,
wherein when a disease onset or progression of which correlates with methylation of the gene is the target disease, the bisulfite reagent is a bisulfite reagent which has been evaluated by the method according to claim 1 as having an inappropriate conversion tendency, and
wherein when a disease onset or progression of which correlates with unmethylation of the gene is the target disease, the bisulfite reagent is a bisulfite reagent which has been evaluated by the method according to claim 1 as having a failed conversion tendency.

3. The method according to claim 2, wherein a false positive rate and a false negative rate in the test are estimated from values of the error rate 1 and the error rate 2 in the evaluation of the bisulfite reagent.

4. The method according to claim 2, wherein the disease is cancer.

5. A method of a genetic test which analyzes a degree of methylation of a gene of a subject who has a history of illness in regard to a target disease onset or progression of which correlates with methylation or unmethylation of the gene, the method comprising treating DNA of the subject with a bisulfite reagent,
wherein when a disease onset or progression of which correlates with methylation of the gene is the target disease, the bisulfite reagent is a bisulfite reagent which has been evaluated by the method according to claim 1 as having a failed conversion tendency, and
wherein when a disease onset or progression of which correlates with unmethylation of the gene is the target disease, the bisulfite reagent is a bisulfite reagent which has been evaluated by the method according to claim 1 as having an inappropriate conversion tendency.

6. The method according to claim 5, wherein a false positive rate and a false negative rate in the test are estimated from values of the error rate 1 and the error rate 2 in the evaluation of the bisulfite reagent.

7. The method according to claim 5, wherein the disease is cancer.
